(19) United States
(12) Patent Application Publication
(10) Pub. No.: US 2026/0021097 A1
(43) Pub. Date: Jan. 22, 2026

(54) COMBINATION THERAPIES COMPRISING A KRAS G12C INHIBITOR AND PEMBROLIZUMAB

(71) Applicants: Genentech, Inc., South San Francisco, CA (US); HOFFMANN-LA ROCHE INC., Little Falls, NJ (US)

(72) Inventors: Ting-Kun Mark LIN, Mill Valley, CA (US); Sandhya Vinayak MANDLEKAR, Foster City, CA (US); Ahmadur RAHMAN, Northolt (GB)

(73) Assignees: Genentech, Inc., South San Francisco, CA (US); HOFFMANN-LA ROCHE INC., Little Falls, NJ (US)

(21) Appl. No.: 19/271,903

(22) Filed: Jul. 17, 2025

Related U.S. Application Data
(60) Provisional application No. 63/672,840, filed on Jul. 18, 2024.

(51) Int. Cl. A61K 31/519; A61K 31/517; A61K 31/573; A61K 39/00; C07K 16/28

(52) U.S. Cl. CPC A61K 31/519; A61K 31/517; A61K 31/573; C07K 16/2818; A61K 2039/545

(57) ABSTRACT

Provided herein are methods of treating KRAS G12C-positive NSCLC by administering a combination of a KRAS G12C inhibitor and pembrolizumab with a prophylactic dose of a steroid.

This application claims benefit of priority to U.S. Application No. 63/672,840 filed Jul. 18, 2024, which is incorporated herein by reference in its entirety.

### FIELD OF INVENTION

Provided herein are combination therapies for treating NSCLC comprising a KRAS G12C inhibitor and pembrolizumab.

### BACKGROUND

Lung cancer remains the leading cause of cancer deaths worldwide and is one of the most common cancers in both men and women (Siegel R L, Miller K D, Fuchs H E, et al. Cancer statistics, 2022. C A Cancer J Clin 2022; 72:7-33.; Torre L A, Siegel R L, Jemal A. Lung Cancer Statistics. Adv Exp Med Biol 2016; 893:1-19.). In 2022 in the United States, it is estimated that there will be 236,740 new cases of lung cancer (117,910 in men and 118,830 in women) and 130,180 lung cancer deaths (American Cancer Society 2022). Data from Europe estimate that in 2022 there will be 244,300 lung cancer deaths (161,700 in men and 82,600 in women) (Dalmartello M, La Vecchia C, Bertuccio P, et al. European cancer mortality predictions for the year 2022 with focus on ovarian cancer. Ann Oncol 2022; 33(3):330-9.).

NSCLC is the predominant subtype of lung cancer, accounting for ˜85% of all cases. NSCLC can be divided into two major histological types: adenocarcinoma and squamous cell carcinoma. Adenocarcinoma histology accounts for more than 50% of all NSCLC cases, while squamous cell histology accounts for approximately 25% of NSCLC.

NSCLC is a heterogeneous disease, and it has become increasingly important to consider molecular biomarker analyses when determining a treatment approach for patients with advanced or metastatic disease. Currently, certain biomarkers are tested for patients having metastatic non-squamous NSCLC. These biomarkers include, for example, epidermal growth factor receptor (EGFR) mutations, anaplastic lymphoma kinase (ALK) rearrangements, BRAF mutations, KRAS mutations, METex14 skipping mutations, NTRK 1/2/3 fusions, RET rearrangements, and ROS1 rearrangements. While being tested, not all of the biomarkers are actionable and each have distinct phenotypic outcomes.

KRAS is a central component of the RAS/mitogen-activated protein kinase (MAPK) signal transduction pathway, an intracellular network of proteins that transmit extracellular growth factor signals to regulate cell proliferation, differentiation, and survival. Oncogenic KRAS mutations that result in the change from glycine to cysteine at position 12 (G12C) are prevalent in NSCLC (˜12%), colorectal cancer (˜4%), and other tumor types (˜4%). The presence of a KRAS mutation in NSCLC has been associated with poor prognosis across the different stages of the disease. Single-agent KRAS G12C inhibitors are currently being established in later lines of treatment for NSCLC but there is no evidence of efficacy in earlier lines of treatment.

PD-L1 expression is also associated with prognosis and efficacy in treating NSCLC and is also recommended to be tested in all patients with advanced or metastatic NSCLC. Immune-mediated adverse reactions can occur at any time after starting treatment with a PD-1/PD-L1 blocking antibody. While immune-mediated adverse events usually manifest during treatment with PD-1/PD-L1 blocking antibodies, immune-mediated adverse events can also manifest after discontinuation of PD-1/PD-L1 blocking antibodies.

In general, advanced stage or metastatic NSCLC remains an incurable disease with limited benefit from the available standard of care treatments, thus demonstrating an unmet medical need in this patient population. Novel combinations incorporating a targeted, biomarker-directed approach may further improve outcomes but are not predictable relative to toxicity profiles and efficacy.

Despite improvements and benefits with CPI therapy in first-line treatment of advanced unresectable or metastatic NSCLC, nearly all patients experience disease progression, including those whose tumor harbors a KRAS G12C mutation. New therapeutic approaches, including CPI and/or targeted therapy combinations, are needed to address this unmet medical need.

Overlapping toxicities of nausea, vomiting, diarrhea, and transaminase elevation with sotorasib, and pembrolizumab were identified in previous clinical trials. (Ernst et. al. Lancet, 102, April 2024, 105074; Li B. T., et al. J. Of Thoracic Onc. 17(9), Supplement, S10-S11.) The severity of adverse events (e.g. Grade 3 and greater), especially transaminase elevation (i.e. Hepatotoxicity can supersede efficacy and prohibit use of the agents in combination. Accordingly, a method of administration is needed to reduce such adverse events when used in combination.

### SUMMARY

Solutions to these and other problems in the art are provided herein.

While current first line treatments for non-small cell lung cancer (NSCLC) include immunotherapy such as pembrolizumab as a single agent or in combination with chemotherapy, most patients experience disease progression. Provided herein are methods that comprise administering to a patient having NSCLC comprising a KRas G12C mutation a combination of a KRAS G12C inhibitor (e.g. divarasib) with pembrolizumab and a prophylactic dose of a steroid (e.g. dexamethasone) as a first line treatment for NSCLC.

The methods provided herein include a prophylactic dose of a steroid in combination with a KRAS G12C inhibitor and pembrolizumab combination. The prophylactic dose of a steroid as described herein reduces the amount and severity of adverse events associated with the combination.

Thus, in one aspect provided herein is a method (M0) of treating KRAS G12C-positive non-small cell lung cancer (NSCLC) in a patient having such NSCLC, where the method comprises administering to the patient an effective amount of a combination comprising:
(a) a KRAS G12C inhibitor;
(b) pembrolizumab; and
(c) a prophylactic dose of a steroid;
wherein the combination is administered as part of a dosing regimen comprising one or more dosing cycles (C), wherein the dosing regimen comprises:
(1) a first KRAS G12C inhibitor cycle (C1) comprising a first dose (C1D1) of the KRAS G12C inhibitor wherein the KRAS G12C inhibitor is administered QD during the cycle; and
(2) a first pembrolizumab cycle (C1) comprising a first dose of pembrolizumab (D1), wherein said pembrolizumab cycle is 21 days (Q3W) or 42 (Q6W) days wherein the pembrolizumab cycle further comprises administration of the prophylactic dose of the steroid administered as (i) a pre-dose one day before the start of the pembrolizumab cycle (C1D⁻1), (ii) a dose on C1D1, and (iii) a dose on the second day of the pembrolizumab cycle (C1D2).

In a second aspect provided herein is a method (M1) of treating KRAS G12C-positive non-small cell lung cancer (NSCLC) in a patient having such NSCLC, the method comprising administering to the patient an effective amount of a combination comprising:
(a) divarasib;
(b) pembrolizumab; and
(c) a prophylactic dose of dexamethasone,
wherein the combination is administered as part of a dosing regimen comprising one or more dosing cycles (C) and wherein the dosing regimen includes a first 21-day cycle (C1) comprising:
(1) a first dose (C1D1) of divarasib administered at an amount of 200 mg or 400 mg, and wherein divarasib is administered at 200 mg or 400 mg QD during the remainder of the cycle; and
(2) a first dose of pembrolizumab (C1D1) administered at an amount of 200 mg, and wherein pembrolizumab is administered Q3W thereafter; and
(3) a prophylactic dose of dexamethasone comprising:
(i) a pre-dose at an effective amount of 4 mg of dexamethasone administered one day before the C1D1 of pembrolizumab (C1D⁻1);
(ii) a dose at an effective amount of 4 mg of dexamethasone administered on C1D1 of the cycle; and
(iii) a dose at an effective amount of 4 mg of dexamethasone administered on the second day of the cycle (C1D2).

In another aspect provided herein is a use of a KRAS G12C inhibitor in the manufacture of a medicament for the treatment of a patient having KRAS G12C-positive non-small cell lung cancer (NSCLC), wherein in said treatment the KRAS G12C inhibitor is to be administered in combination with pembrolizumab and a prophylactic dose of a steroid in a dosing regimen comprising one or more KRAS G12C inhibitor cycles and one or more pembrolizumab cycles.

In another aspect provided herein is a use of a KRAS G12C inhibitor for use in a method of treating a patient having KRAS G12C-positive non-small cell lung cancer (NSCLC), wherein the KRAS G12C inhibitor is to be administered in combination with pembrolizumab and a prophylactic dose of a steroid in a dosing regimen comprising one or more KRAS G12C inhibitor cycles and one or more pembrolizumab cycles.

In still another aspect provided herein is a use of divarasib in the manufacture of a medicament for the treatment of a patient having KRAS G12C-positive non-small cell lung cancer (NSCLC), wherein in said treatment, divarasib is to be administered in combination with pembrolizumab and a prophylactic dose of dexamethasone in a dosing regimen comprising:
(i) a pre-dose at an effective amount of 4 mg of dexamethasone administered one day before the C1D1 of pembrolizumab (C1D⁻1);
(ii) a dose at an effective amount of 4 mg of dexamethasone administered on C1D1 of the cycle; and
(iii) a dose at an effective amount of 4 mg of dexamethasone administered on the second day of the cycle (C1D2), wherein the dosing regimen comprises one or more cycles.

### DETAILED DESCRIPTION

### Definitions

Unless defined otherwise, all technical and scientific terms used herein have the same meaning as commonly understood by those of ordinary skill in the art to which the invention belongs. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention.

The following definitions are provided to facilitate understanding of certain terms used frequently herein and are not meant to limit the scope of the present disclosure. All references referred to herein are incorporated by reference in their entirety.

As used herein, and unless otherwise specified, the terms “about” and “approximately,” when referring to doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. The equivalent dose, amount, or weight percent can be within 30%, 20%, 15%, 10%, 5%, 1%, or less of the specified dose, amount, or weight percent.

“Pembrolizumab” refers to a monoclonal antibody that belongs to a class of drugs that bind to either the programmed death-receptor 1 (PD-1) or the PD-ligand 1 (PD-L1), blocking the PD-1/PD-L1 pathway. Pembrolizumab refers to a humanized monoclonal IgG4 kappa antibody with an approximate molecular weight of 149 kDa. Pembrolizumab can be produced in recombinant Chinese hamster ovary (CHO) cells. Pembrolizumab is commercially marketed as KEYTRUDA®. Pembrolizumab is administered as a sterile injection.

A “KRAS G12C inhibitor” as used herein refers to a compound that is a covalent inhibitor that specifically binds to a mutant KRAS protein comprising a Gly to Cys mutation at a position corresponding to amino acid residue 12.

The term “divarasib” refers to a compound having structure:

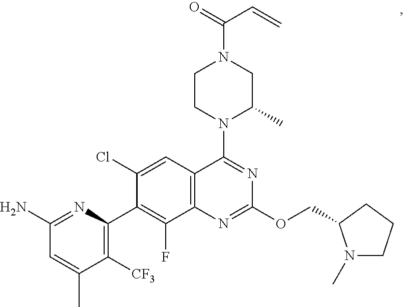

having the chemical name 1-((S)-4-((R)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazin-1-yl)prop-2-en-1-one. In one embodiment, divarasib is an adipate salt. Divarasib is also known as GDC-6036. As used herein, divarasib refers to its freebase and an adipate salt thereof.

“Adagrasib” refers to a compound having structure:

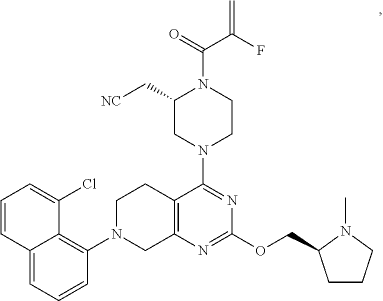 [SMILES: C=C(F)C(=O)N1CCN(c2nc(OC[C@@H]3CCCN3C)nc3c2CCN(c2cccc4cccc(Cl)c24)C3)C[C@@H]1CC#N]

having the chemical name 2-[(2S)-4-[7-(8-chloronaphthalen-1-yl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile.

The term “fulzerasib” refers to a compound having structure:

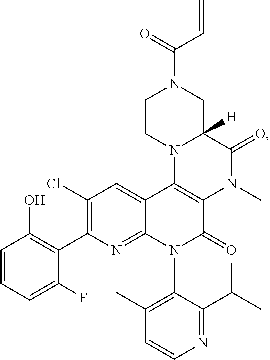 [SMILES: C=CC(=O)N1CCN2c3c(c(=O)n(-c4c(C)ccnc4C(C)C)c4nc(-c5c(O)cccc5F)c(Cl)cc34)N(C)C(=O)[C@H]2C1]

having the chemical name (7R)-16-chloro-15-(2-fluoro-6-hydroxyphenyl)-9-methyl-12-(4-methyl-2-propan-2-ylpyridin-3-yl)-5-prop-2-enoyl-2,5,9,12,14-pentazatetracyclo[8.8.0.02,7.013,18]octadeca-1(10),13,15,17-tetraene-8,11-dione.

The term “garsorasib” refers to a compound having structure:

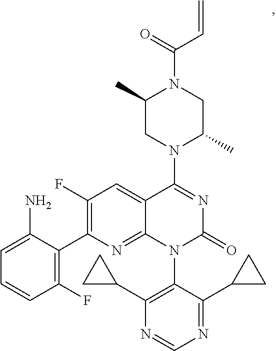 [SMILES: C=CC(=O)N1C[C@H](C)N(c2nc(=O)n(-c3c(C4CC4)ncnc3C3CC3)c3nc(-c4c(N)cccc4F)c(F)cc23)C[C@H]1C]

having the chemical name 7-(2-amino-6-fluorophenyl)-1-(4,6-dicyclopropylpyrimidin-5-yl)-4-[(2S,5R)-2,5-dimethyl-4-prop-2-enoylpiperazin-1-yl]-6-fluoropyrido[2,3-d]pyrimidin-2-one.

The term “glecirasib” refers to a compound having structure:

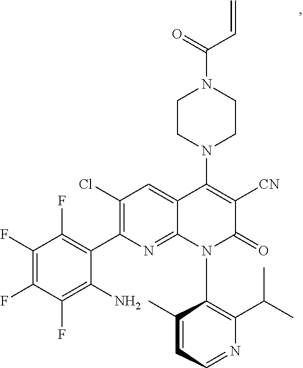

having the chemical name 7-(2-amino-3,4,5,6-tetrafluorophenyl)-6-chloro-1-(4-methyl-2-propan-2-ylpyridin-3-yl)-2-oxo-4-(4-prop-2-enoylpiperazin-1-yl)-1,8-naphthyridine-3-carbonitrile.

The term “olomorasib” refers to a compound having structure:

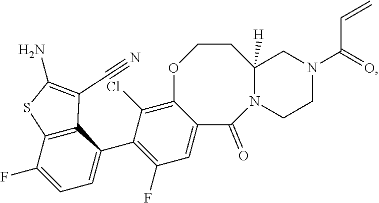

having the chemical name (4R)-2-Amino-4-[(4aS)-8-chloro-10-fluoro-2,3,4,4a,5,6-hexahydro-12-oxo-3-(1-oxo-2-propen-1-yl)-1H,12H-pyrazino[2,1-d][1,5]benzoxazocin-9-yl]-7-fluorobenzo[b]thiophene-3-carbonitrile.

The term “sotorasib” refers to a compound having structure:

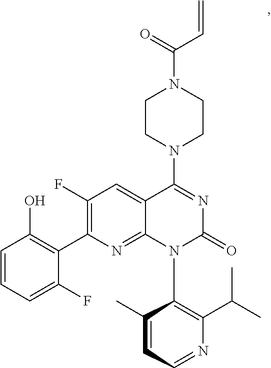

having the chemical name 6-fluoro-7-(2-fluoro-6-hydroxyphenyl)-1-(4-methyl-2-propan-2-ylpyridin-3-yl)-4-[(2S)-2-methyl-4-prop-2-enoylpiperazin-1-yl]pyrido[2,3-d]pyrimidin-2-one.

“Dexamethasone” refers to a synthetic adrenocortical steroid having the structure:

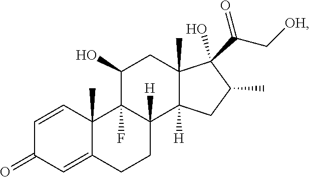

having the chemical name 9-fluoro-11β,17,21trihydroxy-16α-methylpregna-1,4-diene-3,20-dione.

The term “pharmaceutically acceptable” refers to molecular entities and compositions that do not produce an adverse, allergic or other untoward reaction when administered to an animal, such as, for example, a human, as appropriate.

Compounds of the invention may be in the form of a salt, such as a pharmaceutically acceptable salt. The term “pharmaceutically acceptable salt” refers to those salts which retain the biological effectiveness and properties of the free bases and which are not biologically or otherwise undesirable. In one embodiment such salts are formed with inorganic acids such as hydrochloric acid, hydrobromic acid, sulfuric acid, nitric acid, carbonic acid, phosphoric acid and the like. In another embodiment, such salts are formed using organic acids such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, benzenesulfonic acid, p-toluenesulfonic acid, salicylic acid and the like.

In another embodiment, a pharmaceutically acceptable salt is derived from inorganic bases such as sodium, potassium, lithium, ammonium, calcium, magnesium, iron, zinc, copper, manganese, aluminum salts and the like. Particular base addition salts are the ammonium, potassium, sodium, calcium and magnesium salts. In another embodiment, a pharmaceutically acceptable salt derived from pharmaceutically acceptable organic nontoxic bases including, for example, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particular organic non-toxic bases include isopropylamine, diethylamine, ethanolamine, tromethamine, dicyclohexylamine, choline, and caffeine.

The terms “inhibiting” and “reducing,” or any variation of these terms, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity compared to normal.

The terms “antagonist” and “inhibitor” are used interchangeably, and they refer to a compound having the ability to inhibit a biological function of a target protein, whether by inhibiting the activity or expression of the protein, such as K-Ras, H-Ras or N-Ras G12C. Accordingly, the terms “antagonist” and “inhibitors” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g., bind to) the target, compounds that initiate or enhance a biological activity of the target polypeptide by interacting with other members of the signal transduction pathway of which the target polypeptide is a member are also specifically included within this definition.

The term “treatment” refers to clinical intervention designed to alter the natural course of the patient or cell being treated during the course of clinical pathology. Desirable effects of treatment include decreasing the rate of disease progression, ameliorating or palliating the disease state, and remission or improved prognosis. For example, a patient is successfully “treated” if one or more symptoms associated with non-small cell lung cancer (NSCLC) described herein are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of patients. In some embodiments, treatment refers to delaying progression of NSCLC as described herein. In another example, treatment can refer to a reduced level of one or more adverse events associated with one or both of the agents described herein.

The term “delaying progression” of a disease refers to deferring, hindering, slowing, retarding, stabilizing, and/or postponing development of NSCLC as described herein. This delay can be of varying lengths of time, depending on the history of the patient and the disease.

An “effective amount” is at least the minimum amount required to effect a measurable improvement or prevention of NSCLC described herein. An effective amount herein may vary according to factors such as the disease state, age, sex, and weight of the patient, and the ability of the agent to elicit a desired response in the patient. An effective amount is also one in which any toxic or detrimental effects of the treatment are outweighed by the therapeutically beneficial effects. Beneficial or desired results include results such as eliminating or reducing the risk, lessening the severity, delaying the onset of the disease (including biochemical, histological and/or behavioral symptoms of the disease, its complications and intermediate pathological phenotypes presenting during development of the disease), decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In some embodiments, an effective amount of the drug may have the effect in reducing the number of cancer cells; reducing the tumor size; inhibiting (i.e., slow or stop) cancer cell infiltration into peripheral organs; inhibit (i.e., slow or stop) tumor metastasis; inhibiting (i.e., slow or stop) tumor growth; and/or relieving one or more of the symptoms associated with the disease. An effective amount can be administered in one or more administrations. An effective amount of drug, compound, pharmaceutical composition, or combination therapy described herein can be an amount sufficient to accomplish therapeutic treatment either directly or indirectly. As is understood in the clinical context, an effective amount of a drug, compound, or pharmaceutical composition may or may not be achieved in conjunction with another drug, compound, or pharmaceutical composition, or combination therapy. Thus, an effective amount may be considered in the context of administering one or more therapeutic agents, and a single agent may be considered to be given in an effective amount if, in conjunction with one or more other agents, a desirable result may be or is achieved.

A “prophylactic dose” refers to a dose of an agent used to prevent or reduce the effect of or amount of an adverse event described herein. For example, a prophylactic of a steroid described herein may reduce or prevent certain adverse events associated with a KRAS G12C inhibitor or pembrolizumab or the combination thereof (e.g. hepatotoxicity).

“KRAS G12C-positive” refers to a patient who has NSCLC that has been tested for and the presence found of a KRAS G12C mutation.

A “cycle” refers to a period of time comprising administration of one or more agents described herein (e.g. a KRAS G12C inhibitor as described herein and pembrolizumab) and an optional period of time comprising no administration of one or more of the agents described herein. For example, a cycle can be 21 or 28 days in total and include administration of one or more agents described herein (e.g. a KRAS G12C inhibitor as described herein and pembrolizumab) each day of the cycle. In certain instances, the KRAS G12C inhibitor and pembrolizumab may have a different length cycle, such as, for example, a 21 day cycle for pembrolizumab and a 28 day cycle for the KRAS G12C inhibitor. In another example, a cycle can be 28 days in total length and include administration of one or more agents described herein (e.g. a KRAS G12C inhibitor as described herein and pembrolizumab) for 21 days and a rest period of 7 days. A “rest period” refers to a period of time where at least one of the agents described herein (i.e. a KRAS G12C inhibitor as described herein and pembrolizumab) are not administered. In one embodiment, a rest period refers to a period of time where none of the agents described herein (i.e. a KRAS G12C inhibitor as described herein and pembrolizumab) are administered. A rest period as provided herein can in some instances include administration of another agent that is not a KRAS G12C inhibitor as described herein and pembrolizumab. In such instances, administration of another agent during a rest period should not interfere or detriment administration of an agent described herein. In one embodiment, a cycle comprises 21 or 28 days as described herein where the agents are divarasib and pembrolizumab as described herein.

A “dosing regimen” refers to a period of administration of the agents described herein comprising one or more cycles, where each cycle can include administration of the agents described herein at different times or in different amounts.

“QD” refers to administration of an agent described herein once daily.

“BID” refers to administration of an agent described herein twice daily.

“PO” refers to oral administration of an agent described herein.

The term “Q3W” refers to administration of any agent described herein once every three weeks.

The term “Q6W” refers to administration of an agent described herein once every six weeks.

“Adverse event” refers to undesired medical occurrence after exposure to a medicine that is graded using, for example, the grading scale as established by NCI CTCAE. In one embodiment, the adverse event is graded in accordance with the table below.

| Grade | Severity |
|---|---|
| 1 | Mild; asymptomatic or mild symptoms; clinical or diagnostic |
| | observations only; or intervention not indicated |
| 2 | Moderate; minimal, local, or non-invasive intervention |
| | indicated; or limiting age-appropriate instrumental activities |
| | of daily living |
| 3 | Severe or medically significant, but not immediately life- |
| | threatening; hospitalization or prolongation of hospitalization |
| | indicated; disabling; or limiting self-care activities of daily |
| | living |
| 4 | Life-threatening consequences or urgent intervention indicated ^{d} |
| 5 | Death related to adverse event |

### Methods of Treatment and Uses

Provided herein are methods of treating KRAS G12C-positive non-small cell lung cancer (NSCLC) in a patient having NSCLC. In one aspect provided herein is a method of treating KRAS G12C-positive non-small cell lung cancer (NSCLC) in a patient having NSCLC where the method includes administering to the patient an effective amount of a combination comprising:
(a) a KRAS G12C inhibitor;
(b) pembrolizumab; and
(c) a prophylactic dose of a steroid,
   where the combination is administered as part of a dosing regimen comprising one or more dosing cycles (C) and where the dosing regimen includes
(1) a first KRAS G12C inhibitor cycle (C1) comprising a first dose (C1D1) of the KRAS G12C inhibitor where the KRAS G12C inhibitor is administered QD during the cycle; and
(2) a first pembrolizumab cycle (C1) comprising a first dose of pembrolizumab (D1), where said pembrolizumab cycle is 21 days (Q3W) or 42 (Q6W) days where the pembrolizumab cycle further comprises administration of the prophylactic dose of the steroid administered as
(i) a pre-dose one day before the start of the pembrolizumab cycle (C1D⁻1);
(ii) a dose on C1D1; and
(iii) a dose on the second day of the pembrolizumab cycle (C1D2).

In one embodiment of the methods described herein the KRAS G12C inhibitor is sotorasib, adagrasib, glecirasib, olomorasib, garsorasib, fulzerasib, or divarasib, or a pharmaceutically acceptable salt thereof.

In one such embodiment, the KRAS G12C inhibitor is fulzerasib. In such embodiments, fulzerasib is administered orally at an effective amount of 600 mg BID. Fulzerasib can be administered on a 28-day cycle. In another such embodiment, the KRAS G12C inhibitor is garsorasib administered orally at an effective amount of 600 mg BID. Garsorasib can be administered on a 21-day cycle. In another such embodiment, the KRAS G12C inhibitor is glecirasib administered orally at an effective amount of 800 mg QD. In another such embodiment, the KRAS G12C inhibitor is olomorasib administered at an effective amount of 50 mg, 100 mg, or 150 mg BID. In such embodiments, olomorasib can be administered at an effective amount of 50 mg on a 21 or 42 day cycle. Where the KRAS G12C inhibitor is olomorasib as described herein it can be administered at an effective amount of 100 mg on a 21 or 42 day cycle. In still another embodiment, the KRAS G12C inhibitor is adagrasib administered orally at an effective amount 600 mg BID. In an additional embodiment, the KRAS G12C inhibitor is sotorasib administered orally at an effective amount of 240 mg or 960 mg QD. In such embodiments, sotorasib can be administered at an effective amount of 960 mg QD.

In particular embodiments of the methods described herein the KRAS G12C inhibitor is divarasib. In one embodiment, the C1D1 of divarasib is 200 mg or 400 mg. In one such embodiment, the C1D1 of divarasib is 200 mg. In another such embodiment, the C1D1 of divarasib is 400 mg. Divarasib can be administered orally as a tablet or capsule having a strength of 200 mg or 400 mg as described herein.

The methods described herein include a dosing regimen described herein comprising at least one cycle. In certain embodiments the methods described herein include a dosing regimen described herein comprising 2 or more cycles. In one embodiment of the methods described herein, the dosing regimen comprises 1, 2, 3, 4, 5, 6, 7, 8, 9, 10, 11, or 12 dosing cycles. In one embodiment of the methods described herein, the dosing regimen comprises 1-3, 1-4, 1-6, 1-8, 1-12, 1-14, 1-16, 1-18, or 1-24 cycles.

Pembrolizumab is marked as KEYTRUDA® and is approved for use in combination with various agents including (i) pemetrexed and platinum chemotherapy, is indicated for the first-line treatment of patients with metastatic nonsquamous non-small cell lung cancer (NSCLC), with no EGFR or ALK genomic tumor aberrations; (ii) carboplatin and either paclitaxel or paclitaxel protein-bound, is indicated for the first-line treatment of patients with metastatic squamous NSCLC; and (iii) platinum-containing chemotherapy as neoadjuvant treatment, and then continued as a single agent as adjuvant treatment after surgery. The combination of pembrolizumab with a KRAS G12C inhibitor has not been approved for therapeutic use.

Administration of pembrolizumab is via intravenous (IV) injection.

Pembrolizumab can be administered herein according to its approved package insert.

Pembrolizumab is administered using one of two different cycles. In one such cycle, pembrolizumab at an effective amount of 200 mg is administered every 3 weeks (Q3W) or at an effective amount of 400 mg is administered every 6 weeks (Q6W). In one embodiment of the methods described herein, the C1D1 of pembrolizumab is 200 mg administered every 3 weeks (Q3W) on a 21-day cycle. In another embodiment of the methods described herein, the C1D1 of pembrolizumab is 400 mg administered every 6 weeks (Q6W) on a 42-day cycle.

In one embodiment, the methods herein comprise a KRAS G12C inhibitor as described herein and administered in a dosing regimen as described herein in combination with pembrolizumab administered at a C1D1 dose of 200 mg Q3W (every 21 days).

In one such embodiment is a method (MS) of treating KRAS G12C-positive non-small cell lung cancer (NSCLC) in a patient having such NSCLC where the method includes administering to the patient an effective amount of a combination comprising:
(a) sotorasib;
(b) pembrolizumab; and
(c) a prophylactic dose of a steroid (e.g. a steroid from Table 1),
where the combination is administered as part of a dosing regimen comprising one or more dosing cycles (C) and where the dosing regimen includes a first 21-day cycle (C1) comprising:
(1) a first dose (C1D1) of sotorasib administered orally at an effective amount of 240 mg or 960 mg QD, and wherein sotorasib is administered at 240 mg or 960 mg QD during the remainder of the cycle;
(2) a first dose of pembrolizumab (C1D1) administered at an amount of 200 mg, and wherein pembrolizumab is administered Q3W thereafter; and
(3) a prophylactic dose of the steroid comprising:
(i) a pre-dose of the steroid administered at an effective amount set forth in Table 1 and administered one day before the C1D1 of pembrolizumab (C1D⁻1);
(ii) a dose of the steroid administered at an effective amount set forth in Table 1 and administered on C1D1 of the cycle; and
(iii) a dose of the steroid administered at an effective amount set forth in Table 1 and administered on the second day of the cycle (C1D2).

In another such embodiment is a method (MA) of treating KRAS G12C-positive non-small cell lung cancer (NSCLC) in a patient having such NSCLC where the method includes administering to the patient an effective amount of a combination comprising:
(a) adagrasib;
(b) pembrolizumab; and
(c) a prophylactic dose of a steroid (e.g. a steroid from Table 1),
where the combination is administered as part of a dosing regimen comprising one or more dosing cycles (C) and where the dosing regimen includes a first 21-day cycle (C1) comprising:
(1) a first dose (C1D1) of adagrasib administered orally at an effective amount of 600 mg BID, and wherein adagrasib is administered at 600 mg BID during the remainder of the cycle;
(2) a first dose of pembrolizumab (C1D1) administered at an amount of 200 mg, and wherein pembrolizumab is administered Q3W thereafter; and
(3) a prophylactic dose of the steroid comprising:
(i) a pre-dose of the steroid administered at an effective amount set forth in Table 1 and administered one day before the C1D1 of pembrolizumab (C1D⁻1);
(ii) a dose of the steroid administered at an effective amount set forth in Table 1 and administered on C1D1 of the cycle; and
(iii) a dose of the steroid administered at an effective amount set forth in Table 1 and administered on the second day of the cycle (C1D2).

In another such embodiment is a method (M0) of treating KRAS G12C-positive non-small cell lung cancer (NSCLC) in a patient having such NSCLC where the method includes administering to the patient an effective amount of a combination comprising:
(a) olomorasib;
(b) pembrolizumab; and
(c) a prophylactic dose of a steroid (e.g. a steroid from Table 1),
where the combination is administered as part of a dosing regimen comprising one or more dosing cycles (C) and where the dosing regimen includes a first 21-day cycle (C1) comprising:
(1) a first dose (C1D1) of olomorasib administered orally at an effective amount of 50, 100, or 150 mg BID, and wherein olomorasib is administered at 50, 100, or 150 mg BID during the remainder of the cycle;
(2) a first dose of pembrolizumab (C1D1) administered at an amount of 200 mg, and wherein pembrolizumab is administered Q3W thereafter; and
(3) a prophylactic dose of the steroid comprising:
(i) a pre-dose of the steroid administered at an effective amount set forth in Table 1 and administered one day before the C1D1 of pembrolizumab (C1D⁻1);
(ii) a dose of the steroid administered at an effective amount set forth in Table 1 and administered on C1D1 of the cycle; and
(iii) a dose of the steroid administered at an effective amount set forth in Table 1 and administered on the second day of the cycle (C1D2).

In another such embodiment is method (ML) of treating KRAS G12C-positive non-small cell lung cancer (NSCLC) in a patient having such NSCLC where the method includes administering to the patient an effective amount of a combination comprising:
(a) glecirasib;
(b) pembrolizumab; and
(c) a prophylactic dose of a steroid (e.g. a steroid from Table 1),
where the combination is administered as part of a dosing regimen comprising one or more dosing cycles (C) and where the dosing regimen includes a first 21-day cycle (C1) comprising:
(1) a first dose (C1D1) of glecirasib administered orally at an effective amount of 800 mg QD, and wherein glecirasib is administered at 800 mg QD during the remainder of the cycle;
(2) a first dose of pembrolizumab (C1D1) administered at an amount of 200 mg, and wherein pembrolizumab is administered Q3W thereafter; and
(3) a prophylactic dose of the steroid comprising:
(i) a pre-dose of the steroid administered at an effective amount set forth in Table 1 and administered one day before the C1D1 of pembrolizumab (C1D⁻1);
(ii) a dose of the steroid administered at an effective amount set forth in Table 1 and administered on C1D1 of the cycle; and
(iii) a dose of the steroid administered at an effective amount set forth in Table 1 and administered on the second day of the cycle (C1D2).

In another such embodiment is method (MG) of treating KRAS G12C-positive non-small cell lung cancer (NSCLC) in a patient having such NSCLC where the method includes administering to the patient an effective amount of a combination comprising:
(a) garsorasib;
(b) pembrolizumab; and
(c) a prophylactic dose of a steroid (e.g. a steroid from Table 1),
where the combination is administered as part of a dosing regimen comprising one or more dosing cycles (C) and where the dosing regimen includes a first 21-day cycle (C1) comprising:
(1) a first dose (C1D1) of garsorasib administered orally at an effective amount of 600 mg BID, and wherein garsorasib is administered at 600 mg BID during the remainder of the cycle;
(2) a first dose of pembrolizumab (C1D1) administered at an amount of 200 mg, and wherein pembrolizumab is administered Q3W thereafter; and
(3) a prophylactic dose of the steroid comprising:
(i) a pre-dose of the steroid administered at an effective amount set forth in Table 1 and administered one day before the C1D1 of pembrolizumab (C1D⁻1);
(ii) a dose of the steroid administered at an effective amount set forth in Table 1 and administered on C1D1 of the cycle; and
(iii) a dose of the steroid administered at an effective amount set forth in Table 1 and administered on the second day of the cycle (C1D2).

In another such embodiment is method (MF) of treating KRAS G12C-positive non-small cell lung cancer (NSCLC) in a patient having such NSCLC where the method includes administering to the patient an effective amount of a combination comprising:
(a) fulzerasib;
(b) pembrolizumab; and
(c) a prophylactic dose of a steroid (e.g. a steroid from Table 1),
where the combination is administered as part of a dosing regimen comprising one or more dosing cycles (C) and where the dosing regimen includes
(1) a first 28-day cycle (C1) comprising:
(i) a first dose (C1D1) of fulzerasib administered orally at an effective amount of 600 mg BID, and wherein fulzerasib is administered at 600 mg BID during the remainder of the cycle; and
(2) a first 21-day pembrolizumab cycle comprising:
(i) a first dose of pembrolizumab (C1D1) administered at an amount of 200 mg, and wherein pembrolizumab is administered Q3W thereafter; and
(ii) a prophylactic dose of the steroid comprising:
(a) a pre-dose of the steroid administered at an effective amount set forth in Table 1 and administered one day before the C1D1 of pembrolizumab (C1D⁻1); (b) a dose of the steroid administered at an effective amount set forth in Table 1 and administered on C1D1 of the cycle; and
(c) a dose of the steroid administered at an effective amount set forth in Table 1 and administered on the second day of the cycle (C1D2).

In one embodiment, the steroid used in the methods described herein is a steroid as set forth in Table 1.

TABLE 1

| Steroid | Dose (mg) |
|---|---|
| Cortisone | 125 |
| Hydrocortisone | 100 |
| Prednisone | 25 |
| Prednisolone | 25 |
| Triamcinolone | 20 |
| Methylprednisolone | 20 |
| betamethasone | 4 |
| dexamethasone | 4 |

In one embodiment, the steroid of methods M0, MS, MA, MO, ML, MG, and MF as described herein is prednisone, prednisolone, methylprednisolone, betamethasone, or dexamethasone. In another embodiment, the steroid of methods M0, MS, MA, MO, ML, MG, and MF as described herein is prednisone, prednisolone, or methylprednisolone. In one embodiment, the steroid of methods M0, MS, MA, MO, ML, MG, and MF as described herein is triamcinolone or betamethasone.

In one embodiment of the methods described herein, the steroid of methods M0, MS, MA, MO, ML, MG, and MF as described herein is dexamethasone. In some embodiments, dexamethasone is administered orally BID at an amount of about 4 mg on C1D⁻1, C1D1 and C1D2. In one embodiment, dexamethasone is not further administered following C1D2. In one embodiment, dexamethasone is administered as the prophylactic dose with each administration of pembrolizumab. Consequently, each cycle of the dosing regimen comprising pembrolizumab also comprises the prophylactic dose of dexamethasone.

The prophylactic dose of dexamethasone administered on C1D1 and C1D2 can be administered before, concurrently, or after the dose (e.g. C1D1 and QD dose) of the KRAS G12C inhibitor.

In another aspect provided herein is a method (M1) of treating KRAS G12C-positive non-small cell lung cancer (NSCLC) in a patient having such NSCLC where the method includes administering to the patient an effective amount of a combination comprising:
(a) divarasib;
(b) pembrolizumab; and
(c) a prophylactic dose of dexamethasone,
where the combination is administered as part of a dosing regimen comprising one or more dosing cycles (C) and where the dosing regimen includes a first 21-day cycle (C1) comprising:
(1) a first dose (C1D1) of divarasib administered at an amount of 200 mg or 400 mg, and wherein divarasib is administered at 200 mg or 400 mg QD during the remainder of the cycle;
(2) a first dose of pembrolizumab (C1D1) administered at an amount of 200 mg, and wherein pembrolizumab is administered Q3W thereafter; and
(3) a prophylactic dose of dexamethasone comprising:
(i) a pre-dose of dexamethasone administered at an effective amount of 4 mg BID administered one day before the C1D1 of pembrolizumab (C1D⁻1);
(ii) a dose of dexamethasone administered at an effective amount of 4 mg BID administered on C1D1 of the cycle; and
(iii) a dose of dexamethasone administered at an effective amount of 4 mg BID administered on the second day of the cycle (C1D2).

In one embodiment, the NSCLC is advanced or metastatic NSCLS. In such embodiments, the method is for treating NSCLC as a 1L (first line) treatment.

In one embodiment of the method M1 described herein, divarasib is administered at an effective amount of 400 mg. In one such embodiment, the C1D1 of divarasib is 400 mg and the remaining QD administration during the cycle is 400 mg.

In one embodiment of the method M1 described herein, divarasib is administered at an effective amount of 200 mg. In one such embodiment, the C1D1 of divarasib is 200 mg and the remaining QD administration during the cycle is 400 mg.

In one embodiment of the method M1 described herein, divarasib is administered at an effective amount of 400 mg. In one such embodiment, the C1D1 of divarasib is 200 mg and the remaining QD administration during the cycle is 400 mg.

In one embodiment of the method M1 described herein, divarasib is administered at an effective amount of 400 mg. In one such embodiment, the C1D1 of divarasib is 400 mg and the remaining QD administration during the cycle is 200 mg.

In one embodiment, the dose of divarasib can be reduced up to three dose levels. In one such embodiment, the C1D1 of divarasib is 400 mg and is reduced to 200 mg. In another such embodiment, the C1D1 of divarasib is 400 mg, wherein the dose is first reduced to 200 mg followed by a second reduction from 200 mg to 100 mg. In another such embodiment, the C1D1 of divarasib is 400 mg, wherein the dose is first reduced to 200 mg followed by a second reduction from 200 mg to 100 mg followed by a third reduction from 100 mg to 50 mg.

In such embodiments where the dose of divarasib administered to the patient is reduced, re-escalation of the dose may be initiated. Where dose re-escalation in the patient is started, the dose re-escalation follows the same stepwise progression as the reduction. Thus, a patient given a re-escalated dose receives a 100 mg dose (if they were receiving a 50 mg dose); a 200 mg dose (if they were receiving a 100 mg dose); and a 400 mg dose (if they were receiving a 200 mg dose. In such embodiments, the patient cannot receive a skip-level dose increase or more than a single re-escalation dose in a single dosing cycle (C). In such embodiments, the dose of pembrolizumab is not modified.

In one embodiment, the prophylactic dose of dexamethasone is administered with each administration of pembrolizumab. In such embodiments, the prophylactic dose of dexamethasone is administered one day before (C1D⁻1), the day of (C1D1), and one day after (C1D2) the administration of pembrolizumab as described herein.

In one embodiment, the prophylactic dose is administered before, concurrently or after a 200 mg or 400 mg dose of divarasib as described herein. In one such embodiment, the prophylactic dose is administered before divarasib. In one such embodiment, the prophylactic dose is administered after divarasib.

In one embodiment, the efficacy of the combination in method M1 is compared to a standard of care (SOC) treatment. In one such embodiment, the SOC comprises administration of pembrolizumab at 200 mg Q3W in combination with pemetrexed administered at 500 mg/m² Q3W and chemotherapy. In one such embodiment, the chemotherapy comprises administration of either carboplatin (AUC 5 Q3W) or cisplatin (75 mg/mg² Q3W).

In one embodiment, the methods described herein comprising a dosing regimen comprising the administration of a KRAS G12C inhibitor and pembrolizumab with a prophylactic dose of a steroid further comprises reducing Grade 3 or greater hepatotoxicity in the patient comparable to a dosing regimen without administration of the prophylactic dose. In one such embodiment, the KRAS G12C inhibitor is divarasib administered as described herein. In another such embodiment, the KRAS G12C inhibitor is sotorasib or adagrasib administered as described herein or as set forth in the prescribing information. In another such embodiment, the KRAS G12C inhibitor is fulzerasib, garsorasib, glecirasib, or olomorasib administered as described herein or as set forth in the prescribing information. In one such embodiment, the steroid is dexamethasone administered as described herein. In another such embodiment, the steroid is prednisone, prednisolone, or methylprednisolone administered as described herein.

In one such embodiment of the methods described herein, the prophylactic dose of a steroid in the dosing regimen reduces Grade 3 or greater hepatic transaminase elevation comparable to a dosing regimen without administration of the prophylactic dose. In such embodiments, the hepatic transaminase elevation is alanine transaminase (ALT) elevation or aspartate transaminase (AST) elevation, or a combination thereof. In another embodiment of the method M0, the prophylactic dose of a steroid in the dosing regimen reduces Grade 3 or greater nausea, vomiting, or diarrhea comparable to a dosing regimen without administration of the prophylactic dose.

Further provided herein, method M1 comprises the dosing regimen described herein comprising administration of divarasib as described herein and pembrolizumab as described herein with a prophylactic dose of dexamethasone, and further comprises reducing Grade 3 or greater hepatotoxicity in the patient comparable to a dosing regimen without administration of the prophylactic dose of dexamethasone. In one such embodiment of the method M1, the prophylactic dose of a steroid in the dosing regimen reduces Grade 3 or greater of hepatic transaminase elevation comparable to a dosing regimen without administration of the prophylactic dose of dexamethasone. In such embodiments, the hepatic transaminase elevation is alanine transaminase (ALT) elevation or aspartate transaminase (AST) elevation, or a combination thereof. In another embodiment of the method M1, the prophylactic dose of a steroid in the dosing regimen reduces Grade 3 or greater nausea, vomiting, or diarrhea comparable to a dosing regimen without administration of the prophylactic dose of dexamethasone.

In one embodiment of the method M1 described herein, the dose of divarasib administered in the combination is not reduced compared to administration of divarasib as a single agent. In such embodiments, the dose of pembrolizumab administered is also not reduced. As such, method M1 as described herein reduces adverse events associated with the administration of the combination of divarasib and pembrolizumab while not requiring reduction in the dose of either agent. As such, the efficacy of the combination is enhanced comparable to a dosing regimen lacking the prophylactic dose of dexamethasone.

The methods described herein are useful in treating any line of NSCLC (i.e. First line (1L), Second line (2L), or additional lines). In one embodiment, the method of M0 or M1 as described herein includes treatment of 1L NSCLC. In another embodiment, the method of M0 or M1 as described herein includes treatment of 2L NSCLC. In some embodiments, the methods described herein are useful in delaying progression of NSCLC.

### Embodiments

Provided below are exemplary embodiments of the invention.

Embodiment 1. A method of treating KRAS G12C-positive non-small cell lung cancer (NSCLC) in a patient having such NSCLC, the method comprising administering to the patient an effective amount of a combination comprising: (a) a KRAS G12C inhibitor;
(b) pembrolizumab; and
(c) a prophylactic dose of a steroid;
wherein the combination is administered as part of a dosing regimen comprising one or more dosing cycles (C), wherein the dosing regimen comprises:
(1) a first KRAS G12C inhibitor cycle (C1) comprising a first dose (C1D1) of the KRAS G12C inhibitor wherein the KRAS G12C inhibitor is administered QD during the cycle; and
(2) a first pembrolizumab cycle (C1) comprising a first dose of pembrolizumab (D1), wherein said pembrolizumab cycle is 21 days (Q3W) or 42 (Q6W) days wherein the pembrolizumab cycle further comprises administration of the prophylactic dose of the steroid administered as (i) a pre-dose one day before the start of the pembrolizumab cycle (C1D⁻1), (ii) a dose on C1D1, and (iii) a dose on the second day of the pembrolizumab cycle (C1D2).

Embodiment 2. The method of embodiment 1, wherein the KRAS G12C inhibitor is sotorasib, adagrasib, glecirasib, olomorasib, garsorasib, fulzerasib, or divarasib, or a pharmaceutically acceptable salt thereof.

Embodiment 3. The method of embodiment 1, wherein the KRAS G12C inhibitor is divarasib.

Embodiment 4. The method of any one of embodiments 1 to 3, wherein the C1D1 of divarasib is 200 mg or 400 mg.

Embodiment 5. The method of embodiment 4, wherein the C1D1 of divarasib is 400 mg.

Embodiment 6. The method of embodiment 4, wherein the C1D1 of divarasib is 200 mg.

Embodiment 7. The method of any one of embodiments 1 to 6, wherein divarasib is administered at an effective amount of 200 mg or 400 mg QD after the C1D1.

Embodiment 8. The method of embodiment 7, wherein divarasib is administered at an effective amount of 400 mg QD.

Embodiment 9. The method of embodiment 5 or 7, wherein divarasib is administered at an effective amount of 200 mg QD.

Embodiment 10. The method of embodiment 1, wherein the KRAS G12C inhibitor is fulzerasib.

Embodiment 11. The method of embodiment 10, wherein fulzerasib is administered orally at an effective amount of 600 mg BID.

Embodiment 12. The method of embodiment 11, where fulzerasib is administered on a 28 day cycle.

Embodiment 13. The method of embodiment 1, wherein the KRAS G12C inhibitor is garsorasib administered orally at an effective amount of 600 mg BID.

Embodiment 14. The method of embodiment 13, wherein garsorasib is administered on a 21 day cycle.

Embodiment 15. The method of embodiment 1, wherein the KRAS G12C inhibitor is glecirasib administered orally at an effective amount of 800 mg QD.

Embodiment 16. The method of embodiment 1, wherein the KRAS G12C inhibitor is olomorasib administered at an effective amount of 50 mg, 100 mg, or 150 mg BID.

Embodiment 17. The method of embodiment 16, wherein olomorasib is administered at an effective amount of 50 mg on a 21 or 42 day cycle.

Embodiment 18. The method of embodiment 16, wherein olomorasib is administered at an effective amount of 100 mg on a 21 or 42 day cycle

Embodiment 19. The method of embodiment 1, wherein the KRAS G12C inhibitor is adagrasib administered orally at an effective amount 600 mg BID.

Embodiment 20. The method of embodiment 1, wherein the KRAS G12C inhibitor is sotorasib administered orally at an effective amount of 240 mg or 960 mg QD.

Embodiment 21. The method of embodiment 20, wherein sotorasib is administered at an effective amount of 960 mg QD.

Embodiment 22. The method of any one of embodiments 1 to 21, wherein the C1D1 of pembrolizumab is 200 mg administered every 3 weeks (Q3W) or 400 mg administered every 6 weeks (Q6W) during the pembrolizumab cycle.

Embodiment 23. The method of embodiment 22, wherein the C1D1 of pembrolizumab is 200 mg administered every 3 weeks (Q3W) on a 21-day cycle.

Embodiment 24. The method of embodiment 22, wherein the C1D1 of pembrolizumab is 400 mg administered every 6 weeks (Q6W) on a 42-day cycle.

Embodiment 25. The method of any one of embodiments 1 to 24, wherein the steroid is dexamethasone.

Embodiment 26. The method of embodiment 25, wherein dexamethasone is administered orally BID at an amount of about 4 mg on C1D⁻1, C1D1 and C1D2.

Embodiment 27. The method of any one of embodiments 1 to 26, comprising 2 or more cycles.

Embodiment 28. The method of any one of embodiments 1 to 27, wherein dexamethasone is administered as the prophylactic dose with each administration of pembrolizumab.

Embodiment 29. The method of any one of embodiments 1 to 28, wherein the dosing regimen comprises 1, 2, 3, 4, 5, 6, 7, 8, 9, 10, 11, or 12 dosing cycles.

Embodiment 30. The method of any one of embodiments 1 to 28, wherein the dosing regimen comprises 1-3, 1-4, 1-6, 1-8, 1-12, 1-14, 1-16, 1-18, or 1-24 cycles.

Embodiment 31. The method of any one of embodiments 1 to 30, wherein the prophylactic dose of a steroid in the dosing regimen reduces Grade 3 or greater hepatotoxicity in the patient comparable to a dosing regimen without administration of the prophylactic dose.

Embodiment 32. The method of any one of embodiments 1 to 31, wherein the prophylactic dose of a steroid in the dosing regimen reduces levels of Grade 3 or greater level of hepatic transaminase elevation comparable to a dosing regimen without administration of the prophylactic dose.

Embodiment 33. The method of embodiment 32, wherein the hepatic transaminase elevation is alanine transaminase (ALT) elevation or aspartate transaminase (AST) elevation, or a combination thereof.

Embodiment 34. The method of any one of embodiments 1 to 33, wherein the prophylactic dose of a steroid in the dosing regimen reduces Grade 3 or greater nausea, vomiting, or diarrhea.

Embodiment 35. The method of any one of embodiments 1 to 34, wherein the dose of divarasib administered in the combination is not reduced compared to administration of divarasib as a single agent.

Embodiment 36. The method of any one of embodiments 1 to 35, wherein the dose of pembrolizumab administered in the combination is not reduced compared to administration of pembrolizumab as a single agent.

Embodiment 37. A method (M1) of treating KRAS G12C-positive non-small cell lung cancer (NSCLC) in a patient having NSCLC, the method comprising administering to the patient an effective amount of a combination comprising:
(a) divarasib;
(b) pembrolizumab; and
(c) a prophylactic dose of dexamethasone,
wherein the combination is administered as part of a dosing regimen comprising one or more dosing cycles (C) and wherein the dosing regimen includes a first 21-day cycle (C1) comprising:
(1) a first dose (C1D1) of divarasib administered at an amount of 200 mg or 400 mg, and wherein divarasib is administered at 200 mg or 400 mg QD during the remainder of the cycle; and
(2) a first dose of pembrolizumab (C1D1) administered at an amount of 200 mg, and wherein pembrolizumab is administered Q3W thereafter; and
(3) a prophylactic dose of dexamethasone comprising:
(i) a pre-dose of dexamethasone administered at an effective amount of 4 mg BID administered one day before the C1D1 of pembrolizumab (C1D⁻1);
(ii) a dose of dexamethasone administered at an effective amount of 4 mg BID administered on C1D1 of the cycle; and
(iii) a dose of dexamethasone administered at an effective amount of 4 mg BID administered on the second day of the cycle (C1D2).

Embodiment 38. The method of embodiment 37, wherein the C1D1 of divarasib is 400 mg.

Embodiment 39. The method of embodiment 37, wherein the C1D1 of divarasib is 200 mg.

Embodiment 40. The method of any one of embodiments 37 to 39, wherein divarasib is administered QD at an effective amount of 400 mg.

Embodiment 41. The method of any one of embodiments 37 to 39, wherein divarasib is administered QD at an effective amount of 200 mg.

Embodiment 42. The method of any one of embodiments 37 to 41, comprising 2 or more cycles.

Embodiment 43. The method of any one of embodiments 37 to 42, wherein the prophylactic dose of a steroid in the dosing regimen reduces Grade 3 or greater hepatotoxicity in the patient comparable to a dosing regimen without administration of the prophylactic dose.

Embodiment 44. The method of any one of embodiments 37 to 43, wherein the prophylactic dose of a steroid in the dosing regimen reduces Grade 3 or greater hepatic transaminase elevation comparable to a dosing regimen without administration of the prophylactic dose.

Embodiment 45. The method of any one of embodiments 37 to 44, wherein the prophylactic dose of a steroid in the dosing regimen reduces Grade 3 or greater nausea, vomiting, or diarrhea.

Embodiment 46. The method of any one of embodiments 37 to 45, wherein the dose of divarasib and pembrolizumab administered in the combination is not reduced compared to administration of each as a single agent.

Embodiment 47. A kit comprising the combination of embodiment 1 and instructions for use.

Embodiment 48. Use of a KRAS G12C inhibitor in the manufacture of a medicament for the treatment of a patient having a non-small cell lung cancer (NSCLC), wherein in said treatment the KRAS G12C inhibitor is to be administered in combination with pembrolizumab and a prophylactic dose of a steroid in a dosing regimen comprising one or more KRAS G12C inhibitor cycles and one or more pembrolizumab cycles.

Embodiment 49. The use of embodiment 48, wherein the prophylactic dose of the steroid comprises (i) a pre-dose one day before the start of the pembrolizumab cycle, (ii) a dose on the first day of the pembrolizumab cycle, and (iii) a dose on the second day of the pembrolizumab cycle.

Embodiment 50. The use of embodiment 48 or 49, wherein the KRAS G12C inhibitor is sotorasib, adagrasib, glecirasib, olomorasib, garsorasib, fulzerasib, or divarasib, or a pharmaceutically acceptable salt thereof.

Embodiment 51. The use of any one of embodiments 48-50, wherein the KRAS G12C inhibitor is divarasib.

Embodiment 52. The use of any one of embodiments 48 to 51, wherein divarasib is at a dose of about 200 mg or 400 mg.

Embodiment 53. The use of any one of embodiments 48 to 52, wherein pembrolizumab is at a dose of 200 mg Q3W or 400 mg Q6W.

Embodiment 54. The use embodiment 53, wherein pembrolizumab is at a dose of 200 mg Q3W.

Embodiment 55. The use of any one of embodiments 48 to 54, wherein the steroid is dexamethasone.

Embodiment 56. The use of embodiment 55, wherein the dexamethasone is at a dose of about 4 mg.

Embodiment 57. A KRAS G12C inhibitor for use in a method of treating a patient having non-small cell lung cancer (NSCLC), wherein the KRAS G12C inhibitor is to be administered in combination with pembrolizumab and a prophylactic dose of a steroid in a dosing regimen comprising one or more KRAS G12C inhibitor cycles and one or more pembrolizumab cycles.

Embodiment 58. The KRAS G12C inhibitor of embodiment 57, wherein the prophylactic dose of the steroid comprises (i) a pre-dose one day before the start of the pembrolizumab cycle, (ii) a dose on the first day of the pembrolizumab cycle, and (iii) a dose on the second day of the pembrolizumab cycle.

Embodiment 59. The KRAS G12C inhibitor of embodiment 57 or 58, wherein the KRAS G12C inhibitor is sotorasib, adagrasib, glecirasib, olomorasib, garsorasib, fulzerasib, or divarasib, or a pharmaceutically acceptable salt thereof.

Embodiment 60. The KRAS G12C inhibitor of any one of embodiments 57-59, wherein the KRAS G12C inhibitor is divarasib.

Embodiment 61. The KRAS G12C inhibitor of any one of embodiments 57-60, wherein divarasib is at a dose of about 200 mg or 400 mg.

Embodiment 62. The KRAS G12C inhibitor of any one of embodiments 57-61, wherein pembrolizumab is at a dose of 200 mg Q3W or 400 mg Q6W.

Embodiment 63. The KRAS G12C inhibitor embodiment 62, wherein pembrolizumab is at a dose of 200 mg Q3W.

Embodiment 64. The KRAS G12C inhibitor of any one of embodiments 57-63, wherein the steroid is dexamethasone.

Embodiment 65. The KRAS G12C inhibitor of embodiment 64, wherein the dexamethasone is at a dose of about 4 mg.

Embodiment 66. Use of divarasib in the manufacture of a medicament for the treatment of a patient having KRAS G12C-positive non-small cell lung cancer (NSCLC), wherein in said treatment divarasib is to be administered in combination with pembrolizumab and a prophylactic dose of dexamethasone in a dosing regimen comprising:
(i) a pre-dose at an effective amount of 4 mg of dexamethasone administered one day before the C1D1 of pembrolizumab (C1D⁻1);
(ii) a dose at an effective amount of 4 mg of dexamethasone administered on C1D1 of the cycle; and
(iii) a dose at an effective amount of 4 mg of dexamethasone administered on the second day of the cycle (C1D2), wherein the dosing regimen comprises one or more cycles.

Embodiment 67. The use of embodiment 66, wherein divarasib is at a dose of about 200 mg or 400 mg.

Embodiment 68. The use of embodiment 66 or 67, wherein divarasib is at a dose of about 400 mg.

Embodiment 69. The use of embodiment 66 or 67, wherein divarasib is at a dose of about 200 mg.

Embodiment 70. The use of any one of embodiments 66-69, wherein pembrolizumab is at a dose of 200 mg Q3W or 400 mg Q6W.

Embodiment 71. The use of embodiment 70, wherein pembrolizumab is at a dose of 200 mg Q3W.

Embodiment 72. Divarasib for use in a method of treating a patient having non-small cell lung cancer (NSCLC), wherein divarasib is to be administered in combination with pembrolizumab and a prophylactic dose of dexamethasone in a dosing regimen comprising:
(i) a pre-dose at an effective amount of 4 mg of dexamethasone administered one day before the C1D1 of pembrolizumab (C1D⁻1);
(ii) a dose at an effective amount of 4 mg of dexamethasone administered on C1D1 of the cycle; and
(iii) a dose at an effective amount of 4 mg of dexamethasone administered on the second day of the cycle (C1D2), wherein the dosing regimen comprises one or more cycles.

Embodiment 73. The compound of embodiment 72, wherein divarasib is at a dose of about 200 mg or 400 mg.

Embodiment 74. The use of embodiment 72 or 73, wherein divarasib is at a dose of about 400 mg.

Embodiment 75. The use of embodiment 72 or 73, wherein divarasib is at a dose of about 200 mg.

Embodiment 76. The use of any one of embodiments 72-75, wherein pembrolizumab is at a dose of 200 mg Q3W or 400 mg Q6W.

Embodiment 77. The use of embodiment 76, wherein pembrolizumab is at a dose of 200 mg Q3W.

Embodiment 78. The invention as described hereinbefore.

### Examples

The development of combination treatments poses challenges including, for example, the selection of agents for combination therapy that may lead to improved efficacy while maintaining acceptable toxicity. One particular challenge is the need to distinguish the incremental toxicity of the combination and/or exacerbation of adverse events in combination.

Combination therapies are known to exacerbate existing adverse events or result in new adverse events. Many adverse events are known to result in interruption of pembrolizumab administration. The most reported adverse events of administering pembrolizumab were neutropenia (13%), asthenia/fatigue (7%), anemia (7%), thrombocytopenia (5%), diarrhea (4%), pneumonia (4%), increased blood creatinine (3%), dyspnea (2%), febrile neutropenia (2%), upper respiratory tract infection (2%), increased ALT (2%), and pyrexia (2%).

It is generally understood that the when an adverse event occurs, four options exist: (1) continue treatment as-is with optional concomitant therapy; (2) adjust the dose of one or more agents in the dosing regimen; (3) suspend administration of one or more agents in the dosing regimen; or (4) discontinue administration of one or more agents in the dosing regimen. Adjusting dose, suspending administration, and/or discontinuing administration effects the efficacy of the combination. And often, concomitant therapy can be insufficient to reduce severe adverse events. In such instances, such combinations are insufficiently efficacious to continue development.

Although first-line treatments with pembrolizumab in combination with platinum-based chemotherapy were found to improve OS for patients with advanced or metastatic NSCLC, such treatments are less efficacious in patient populations having oncogenic driver mutations such as KRAS G12C mutations. The novel combinations and prophylactic steroid doses described herein offer a chemotherapy-free regimen that has greater efficacy, avoids chemotherapy-related toxicities, and improves long-term patient outcomes and quality of life.

Checkpoint-inhibitor immunotherapy is associated with hepatotoxicity.

However, such hepatotoxicity is usually Grade 3 or lower and occurs in only about 3-4% of patients. However, severe hepatic transaminase elevation is known as a serious adverse event for the combination of oral KRAS G12C inhibitors currently in clinical development with immunotherapy (e.g. Pembrolizumab). (Skoulidis et al. N Engl J Med 2021; 384:2371-81.; Jänne et al. 2020.) An association between PD-(L)1 inhibitor administration and sotorasib-related hepatic transaminase elevation was identified. (Begum et al. 2021; Chour et al. 2022; Rakshit et al. 2022.) Treatment-related hepatic transaminase elevations (including Grade 3 adverse events) have been reported with both sotorasib and adagrasib in clinical trials. (Skoulidis et al. 2021; Jänne et al. 2020.) Patients taking pembrolizumab also have known risk for immune-mediated myocarditis. Divarasib has not been shown to accentuate the risk.

Concurrent or lead-in sotorasib and PD(L)1 combination resulted in a higher incidence of Grade 3-4 treatment-related hepatotoxicity than observed with single-agent sotorasib (Li et al. 2022). The CodeBreaK 100/101 phase 1 b dose exploration trial was performed with sotorasib in combination with pembrolizumab without any administration of a steroid. The resulting data showed that the patients in the CodeBreaK 100/101 trial presented a statistically higher incidence of Grade 3-4 adverse events, predominantly hepatotoxicity, particularly when sotorasib was concurrently administered in combination with pembrolizumab or atezolizumab (74% with pembrolizumab and 50% with atezolizumab). (Ernst et al.) A total of 65% of the patients administered sotorasib in combination with pembrolizumab experienced hepatotoxicity, of whom 31% experienced severe hepatotoxicity. Among these patients, five were hospitalized during the severe hepatotoxicity, of whom four patients presented with additional clinical problems that warranted hospitalization. (Ernst et al.) The hepatotoxicity was considered unmanageable and the continuation of development of the combination was stopped due to the severity of hepatotoxicity and other adverse events.

Likewise, treatment-related hepatotoxicity was observed when using adagrasib and pembrolizumab in combination. The dose of adagrasib was lowered to try to reduce the hepatotoxicity associated with the combination. (Jänne et al. N Engl J Med 2022; 387:120-31.) There is significant need to develop specific oncogenic driver therapies that can be managed safely in combination with pembrolizumab.

Even when patients received previous anti-PD-(L)1 therapy (e.g. 56 weeks between anti-PD-(L)1 and sotorasib), such patients still displayed severe hepatotoxicity. (Ernst et al.) Prior anti-PD-(L)1 administration was, therefore, associated with a significantly higher incidence of severe hepatotoxicity (35%). (Id.) And, patients who experienced immune-related hepatotoxicity during prior anti-PD-(L)1 treatment, had a greater incidence of severe hepatotoxicity when administering sotorasib in combination with pembrolizumab (75%). (Id.) It should be noted that in patients with advanced NSCLC receiving immune checkpoint blockade monotherapy experienced only 3.9% grade ≥3 ALT or AST increase.

It was discovered herein that patients treated with divarasib and pembrolizumab in combination and without the prophylactic dose of a steroid as described herein also experienced Grade 3 or higher adverse events including severe hepatotoxicity and nausea, diarrhea and vomiting (N/D/V). To overcome these adverse events, especially severe hepatotoxicity, it was discovered herein that the administration of a prophylactic dose of a steroid at the start of the cycle reduced Grade 3-4 hepatotoxicity and N/D/V. This therapy provides patients with a chemotherapy-free regimen that has greater efficacy, avoids chemotherapy-related toxicities, and improves long-term patient outcomes and quality of life.

As the majority of patients who are eligible for treatment with KRAS G12C inhibitors, including divarasib, receive upfront anti-PD-(L)1 treatment with, for example, pembrolizumab, the surprising efficacy of the prophylactic dose of a steroid described herein opened a path to safely administer KRAS G12C inhibitors, such as divarasib, in combination with checkpoint inhibitors such as pembrolizumab, reducing or reversing the severity of the adverse events associated with the combination.

Table 2 compares treatment outcome and adverse events of patients (N=19) that received the prophylactic dose of dexamethasone described herein to patients (N=35) that were not administered the prophylactic dose of dexamethasone. Patients who received the prophylactic dose of dexamethasone as described herein experienced less Grade 3 nausea, vomiting, diarrhea, AST increased or ALT increased events (51.4% [ 18/35] vs 5.3% [ 1/19]).

Furthermore, as shown by Table 2, dose interruption or reductions were completely eliminated, therefore allowing for the greatest efficacy of the combination described herein. The amount of all adverse events fell compared to regimens lacking the prophylactic dose of dexamethasone. The prophylactic dose of dexamethasone reduced the incidence of Grade 3 or higher hepatotoxicity by over 30%.

TABLE 2
Treatment emergent AEs

| | | |
|---|---|---|
| All | | No |
| Patients | Dexamethasone | Dexamethasone |
| (n = 54) | (n = 19) | (n = 35) |

Overall AEs

| | | | | | | |
|---|---|---|---|---|---|---|
| Any Grade AEs | 48 | (88.9%) | 14 | (73.7%) | 34 | (97.1%) |
| Any Grade AST/ALT inc | 23 | (42.6%) | 4 | (21.1%) | 21 | (60%) |
| AEs | | | | | | |
| Any Grade GI (N/V/D) | 42 | (77.8%) | 11 | (57.9%) | 31 | (88.6%) |
| AEs | | | | | | |
| AEs leading to Diva dose | 26 | (48.1%) | 4 | (21.1%) | 22 | (62.9%) |
| interrupt | | | | | | |
| AEs leading to Pembro | 22 | (40.7%) | 5 | (26.3%) | 17 | (48.6%) |
| dose interrupt | | | | | | |
| AEs leading to Diva dose | 14 | (25.9%) | 1 | (5.3%) | 13 | (37.1%) |
| reduction | | | | | | |
| AEs leading to Diva | 4 | (7.4%) | 1 | (5.3%) | 3 | (8.6%) |
| withdrawal | | | | | | |
| AEs leading to Pembro | 5 | (9.3%) | 1 | (5.3%) | 4 | (11.4%) |
| withdrawal | | | | | | |

Overall AESIs

| All AESIs (>Gr3 N/V/D | 19 | (35.2%) | 1 | (5.3%) | 18 | (51.4%) |
|---|---|---|---|---|---|---|
| or AST/ALT inc AEs) | | | | | | |
| AESIs leading to Diva | 14 | (25.9%) | 0 | | 14 | (40%) |
| dose interrupt | | | | | | |
| AESIs leading to Pembro | 12 | (22.2%) | 0 | | 12 | (34.3%) |
| dose interrupt | | | | | | |
| AESIs leading to Diva | 9 | (16.7%) | 0 | | 9 | (25.7%) |
| dose reduction | | | | | | |
| AESIs leading to Diva | 3 | (5.6%) | 1 | (5.3%) | 2 | (5.7%) |
| withdrawal | | | | | | |
| AESIs leading to Pembro | 3 | (5.6%) | 1 | (5.3%) | 2 | (5.7%) |
| withdrawal | | | | | | |

GI (Gr ≥3 N/V/D) AESIs

| GI AESIs | 9 | (16.7%) | 0 | 9 | (25.7%) |
|---|---|---|---|---|---|
| GI AESIs leading to | 6 | (11.1%) | 0 | 6 | (17.1%) |
| Diva dose interrupt | | | | | |
| GI AESIs leading to | 3 | (5.6%) | 0 | 3 | (8.6%) |
| Pembro dose interrupt | | | | | |
| GI AESIs leading to | 1 | (1.9%) | 0 | 1 | (2.9%) |
| Diva dose reduction | | | | | |
| GI AESIs leading to | 0 | | 0 | 0 | |
| Diva withdrawal | | | | | |
| GI AESIs leading to | 0 | | 0 | 0 | |
| Pembro withdrawal | | | | | |

AST/ALT Inc (Gr ≥3) AESIs

| AST/ALT Inc AESIs | 14 | (25.9%) | 1 | (5.3%)c | 13 | (37.1%) |
|---|---|---|---|---|---|---|
| AST/ALT Inc AESIs | 8 | (14.8%) | 0 | | 8 | (22.9%) |
| leading to Diva dose | | | | | | |
| interrupt | | | | | | |
| AST/ALT Inc AESIs | 9 | (16.7%) | 0 | | 9 | (25.7%) |
| leading to Pembro dose | | | | | | |
| interrupt | | | | | | |
| AST/ALT Inc AESIs | 8 | (14.8%) | 0 | | 8 | (22.9%) |
| leading to Diva dose | | | | | | |
| reduction | | | | | | |
| AST/ALT Inc AESIs | 3 | (5.6%) | 1 | (5.3%) | 2 | (5.7%) |
| leading to Diva | | | | | | |
| withdrawal | | | | | | |
| AST/ALT Inc AESIs | 3 | (5.6%) | 1 | (5.3%) | 2 | (5.7%) |
| leading to Pembro | | | | | | |
| withdrawal | | | | | | |

AESI = Adverse event of special interest;
GI = gastrointestinal;
N/V/D = nausea/vomiting/diarrhea

Given the half-life differential of pembrolizumab and dexamethasone, it was surprising that a 3-day prophylactic dose sufficiently reduced hepatotoxicity associated with the combination of a KRAS G12C inhibitor and pembrolizumab. Consequently, the administration of a prophylactic dose of a steroid (e.g. Dexamethasone) surprisingly and significantly reduced the amount and severity of adverse events for administration of a KRAS G12C inhibitor (e.g. Divarasib) and pembrolizumab.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method of treating KRAS G12C-positive non-small cell lung cancer (NSCLC) in a patient having such NSCLC, the method comprising administering to the patient an effective amount of a combination comprising:
(a) a KRAS G12C inhibitor;
(b) pembrolizumab; and
(c) a prophylactic dose of a steroid;
wherein the combination is administered as part of a dosing regimen comprising one or more dosing cycles (C), wherein the dosing regimen comprises:
(1) a first KRAS G12C inhibitor cycle (C1) comprising a first dose (C1D1) of the KRAS G12C inhibitor wherein the KRAS G12C inhibitor is administered QD during the cycle; and
(2) a first pembrolizumab cycle (C1) comprising a first dose of pembrolizumab (D1), wherein said pembrolizumab cycle is 21 days (Q3W) or 42 (Q6W) days wherein the pembrolizumab cycle further comprises administration of the prophylactic dose of the steroid administered as (i) a pre-dose one day before the start of the pembrolizumab cycle (C1D⁻1), (ii) a dose on the first day of the pembrolizumab ccle C1D1, and (iii) a dose on the second day of the pembrolizumab cycle (C1D2).

2. The method of claim 1, wherein the KRAS G12C inhibitor is sotorasib, adagrasib, glecirasib, olomorasib, garsorasib, fulzerasib, or divarasib, or a pharmaceutically acceptable salt thereof.

3. The method of claim 1, wherein the KRAS G12C inhibitor is divarasib.

4. The method of claim 3, wherein the C1D1 of divarasib is 200 mg or 400 mg.

5. The method of claim 4, wherein the C1D1 of divarasib is 400 mg.

6. The method of claim 4, wherein divarasib is administered at an effective amount of 200 mg or 400 mg QD after the C1D1.

7. The method of claim 5, wherein divarasib is administered at an effective amount of 400 mg QD.

8. The method of claim 5, wherein divarasib is administered at an effective amount of 200 mg QD.

9. The method of claim 1, wherein the KRAS G12C inhibitor is fulzerasib administered orally at an effective amount of 600 mg BID.

10. The method of claim 2, wherein the KRAS G12C inhibitor is garsorasib administered orally at an effective amount of 600 mg BID.

11. The method of claim 2, wherein the KRAS G12C inhibitor is glecirasib administered orally at an effective amount of 800 mg QD.

12. The method of claim 2, wherein the KRAS G12C inhibitor is olomorasib administered at an effective amount of 50 mg, 100 mg, or 150 mg BID.

13. The method of claim 2, wherein the KRAS G12C inhibitor is adagrasib administered orally at an effective amount 600 mg BID.

14. The method of claim 2, wherein the KRAS G12C inhibitor is sotorasib administered orally at an effective amount of 240 mg or 960 mg QD.

15. The method of claim 1, wherein the C1D1 of pembrolizumab is 200 mg administered every 3 weeks (Q3W) or 400 mg administered every 6 weeks (Q6W) during the pembrolizumab cycle.

16. The method of claim 1, wherein the C1D1 of pembrolizumab is 200 mg administered every 3 weeks (Q3W) on a 21-day cycle.

17. The method of claim 1, wherein the steroid is dexamethasone.

18. The method of claim 17, wherein dexamethasone is administered orally BID at an amount of about 4 mg on C1D⁻1, C1D1 and C1D2.

19. The method of claim 17, wherein dexamethasone is administered as the prophylactic dose with each administration of pembrolizumab.

20. (canceled)

21. The method of claim 1, wherein the prophylactic dose of a steroid in the dosing regimen reduces Grade 3 or greater hepatotoxicity, nausea, vomiting, or diarrhea, or a combination thereof in the patient comparable to a dosing regimen without administration of the prophylactic dose.

22. The method of claim 1, wherein the prophylactic dose of a steroid in the dosing regimen reduces levels of Grade 3 or greater level of hepatic transaminase elevation, wherein the hepatic transaminase elevation is alanine transaminase (ALT) elevation or aspartate transaminase (AST) elevation, or a combination thereof, comparable to a dosing regimen without administration of the prophylactic dose or to a standard of care (SOC) therapy.

23. (canceled)

24. (canceled)

25. The method of claim 4, wherein the dose of divarasib administered in the combination is not reduced compared to administration of divarasib as a single agent.

26. A method (M1) of treating KRAS G12C-positive non-small cell lung cancer (NSCLC) in a patient having such NSCLC, the method comprising administering to the patient an effective amount of a combination comprising:
(a) divarasib;
(b) pembrolizumab; and
(c) a prophylactic dose of dexamethasone,
wherein the combination is administered as part of a dosing regimen comprising one or more dosing cycles (C) and wherein the dosing regimen includes a first 21-day cycle (C1) comprising:
(1) a first dose (C1D1) of divarasib administered at an amount of 200 mg or 400 mg, and wherein divarasib is administered at 200 mg or 400 mg QD during the remainder of the cycle;
(2) a first dose of pembrolizumab (C1D1) administered at an amount of 200 mg, and wherein pembrolizumab is administered Q3W thereafter; and
(3) a prophylactic dose of dexamethasone comprising:
(i) a pre-dose of dexamethasone administered at an effective amount of 4 mg BID administered one day before the C1D1 of pembrolizumab (C1D⁻1);
(ii) a dose of dexamethasone administered at an effective amount of 4 mg BID administered on C1D1 of the cycle; and
(iii) a dose of dexamethasone administered at an effective amount of 4 mg BID administered on the second day of the cycle (C1D2).

27. The method of claim 26, wherein the C1D1 of divarasib is 400 mg.

28. The method of claim 27, wherein divarasib is administered QD thereafter at an effective amount of 400 mg.

29. The method of claim 26, wherein the NSCLC is non-squamous advanced or metastatic NSCLC.

30.-32. (canceled)

33. The method of claim 26, wherein the prophylactic dose of dexamethasone reduces Grade 3 or greater hepatotoxicity, nausea, vomiting, or diarrhea, or a combination thereof in the patient comparable to a dosing regimen without administration of the prophylactic dose of dexamethasone.

34. The method of claim 26, wherein the prophylactic dose of dexamethasone reduces levels of Grade 3 or greater level of hepatic transaminase elevation comparable to a dosing regimen without administration of the prophylactic dose of dexamethasone.

35. (canceled)
